(19) United States
(12) Patent Application Publication
(10) Pub. No.: US 2024/0404526 A1
(43) Pub. Date: Dec. 5, 2024

(54) System and Method Using Speech-to-Text Artificial Intelligence to Transcribe a Doctor-Patient Interaction Into a Text Form

(71) Applicant: Alexander Pearson Sheppert, Vancouver, WA (US)

(72) Inventor: Alexander Pearson Sheppert, Vancouver, WA (US)

(21) Appl. No.: 18/733,702

(22) Filed: Jun. 4, 2024

Related U.S. Application Data
(60) Provisional application No. 63/471,071, filed on Jun. 5, 2023.

(51) Int. Cl. G10L 15/26; G16H 80/00

(52) U.S. Cl. CPC G10L 15/26; G16H 80/00

(57) ABSTRACT

A system and method using speech-to-text artificial intelligence to transcribe a doctor-patient interaction into a text format. A website or application on a computer, phone, or device records an interaction. A speech-to-text artificial intelligence that will transcribe the doctor-patient interaction into a text format. After the system of the present invention has received the transcription, it will ask the doctor what sections he would like in his medical note. After a selection of the pieces of the note desired, the transcription of the recording between the doctor and patient is sent to the application server. The application server will use a large-language model AI to determine the content of any of the medical note sections. This input length is almost universally significantly shorter than the length of a standard medical interaction. The application uses three techniques to create a single note from the input.

### FEDERALLY SPONSORED RESEARCH

Not Applicable

### SEQUENCE LISTING OR PROGRAM

Not Applicable

### TECHNICAL FIELD OF THE INVENTION

The present invention relates in general to electronic medical records. More specifically, the present invention relates to automatic generation of medical records, specifically doctor-patient communication notes.

### BACKGROUND OF THE INVENTION

Speech recognition systems for medical reporting have been available commercially for over two decades. Speech recognition is an input mechanism available to assist with clinical documentation by translating speech into text, or verbally controlling user interface functions. Speech recognition has been adopted successfully in limited clinical settings; however, speech recognition has not been uniformly used across all clinical domains. In contrast, speech recognition is now widely used in many consumer applications, including interface control and question answering applications in smart phones.

Early adoption of speech recognition-based documentation was hindered by immature technology and clinically unacceptable recognition error rates, but steady advances in recognition algorithm design and system performance have been made over the years. In particular, the underlying technology within speech recognition systems has evolved dramatically with advances in both the speech recognition engines used to recognize speech, as well as the speed and memory of the hardware used to process speech data.

With the current rapidly developing text based artificial intelligence (AI) systems becoming increasingly available, many of the shortcomings of immature technology are rapidly being overcome.

Therefore, what is needed is a system and method to quickly apply and distribute access to these new technologies and AI language models in medical settings. The present invention teaches the use of existing hardware microphones in devices such as desktop, laptop, tablets, and mobile electronic devices such as smartphones that have become a fixture in medical examination rooms.

Using the existing hardware, the present invention proposes a solution to the access problem and providing connectivity to a large language model AI in processing and creating medial notes from recorded doctor-patient interactions, which represents an improvement over existing AI systems and an advantage in accessibility and usage.

### SUMMARY OF THE INVENTION

The present invention is a system and method using speech-to-text artificial intelligence to transcribe a doctor-patient interaction into a text format. The present invention teaches an AI system created to help doctors with medical documentation.

A doctor opens a website or application on their computer, phone, or device. After logging in, the doctor will press “record” as he begins to interact with a patient. The doctor's device will record the interaction. The website or application uses a speech-to-text artificial intelligence that will transcribe the doctor-patient interaction into a text format. The system of the present invention will switch on and off which speech-to-text AI that it uses.

After the system of the present invention has received the transcription, it will ask the doctor what sections he would like in his medical note. After the doctor has made a selection of the pieces of the note he would like, the transcription of the recording between the doctor and patient is sent to the application server. The application server will use a large-language model AI to determine the content of any of the medical note sections.

One of the challenges in using an LLM to determine medical section information from a medical transcript is that most LLMs of necessity due to the way they operate with transformers and are trained will have a maximum input length of tokens. This input length is almost universally significantly shorter than the length of a standard medical interaction.

To get around this the application uses three techniques. All three of these methods first involve splitting the transcription into chunks that are shorter than the token limit.

In a first method, the method and system of the present invention will use the AI to write a note for each section as if the chunk were the entirety of the visit. Concatenate these notes into a single string and pass this string back into the LLM with the instruction to create a single note out of the many.

In a second method, the method and system of the present invention will use the AI to summarize the medically relevant portions of the chunks and then use the summarized medical portions as the input for the original AI to write the sections on the whole note.

In a third method, the method and system of the present invention will use the AI to extract the medically relevant information with regards to each section of the medical note i.e., “surgical history” from the encounter, and then use the original AI to write sections based on the chunked summaries.

### BRIEF DESCRIPTION OF THE DRAWINGS

The accompanying drawings, which are incorporated herein and form a part of the specification, illustrate the present invention and, together with the description, further explain the principles of the invention and to enable a person skilled in the pertinent art to make and use the invention.

FIG. 1 is a flow chart illustrating the basic process taught by the present invention.

FIG. 2 is a flow chart illustrating the three methods taught by the present invention for first involve splitting the transcription into chunks that are shorter than the token limit.

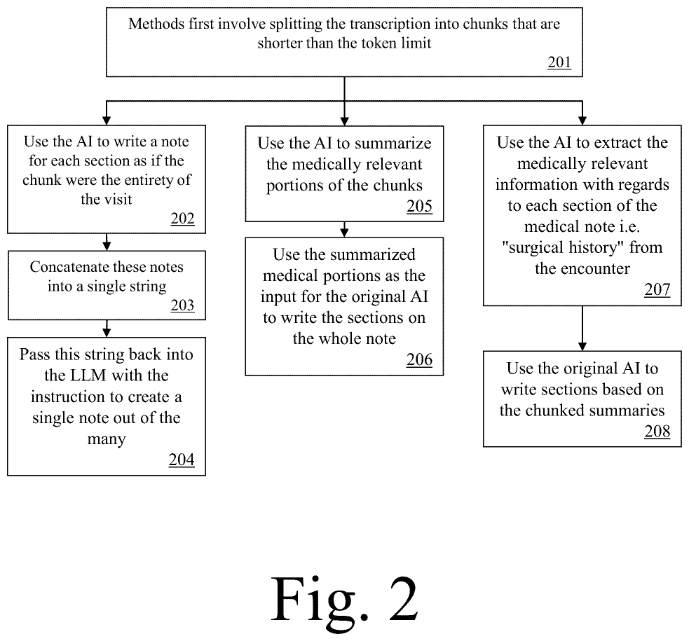

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized, and logical, mechanical, electrical, and other changes may be made without departing from the scope of the present invention. The following detailed description is, therefore, not to be taken in a limiting sense, and the scope of the present invention is defined only by the appended claims.

In the following description, specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figure, it is possible to see the various major elements constituting the apparatus of the present invention.

The present invention is a system and method using speech-to-text artificial intelligence to transcribe a doctor-patient interaction into a text format. The present invention teaches an AI system created to help doctors with medical documentation.

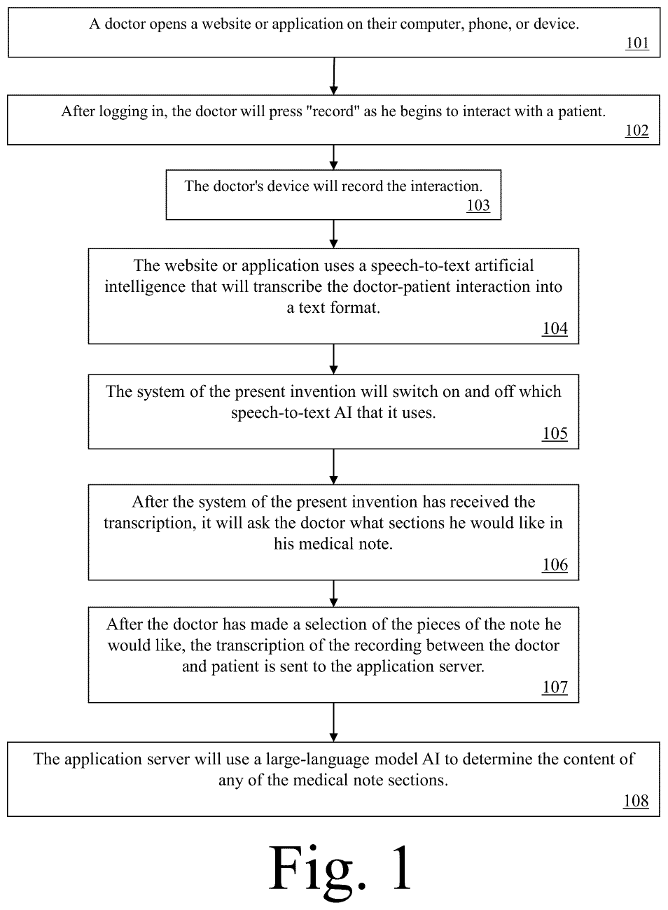

Now referring to FIG. 1, a flow chart of the basic process taught by the present invention is illustrated. In a first step, a doctor opens a website or application on their computer, phone, or device. After logging in, the doctor will press “record” as he begins to interact with a patient. The doctor's device will record the interaction. The website or application uses a speech-to-text artificial intelligence that will transcribe the doctor-patient interaction into a text format. The system of the present invention will switch on and off which speech-to-text AI that it uses.

After the system of the present invention has received the transcription, it will ask the doctor what sections he would like in his medical note. These sections may include but are not limited to: Chief Complaint, History of Present Illness, Past Medical History, Review of Systems, Family History, Social History, Medication List, Physical Examination/Objective section, Assessment or Diagnosis, Plan, and Notes.

After the doctor has made a selection of the pieces of the note he would like, the transcription of the recording between the doctor and patient is sent to the application server. The application server will use a large-language model AI (can use any number of LLMs) to determine the content of any of the medical note sections.

One of the challenges in using an LLM to determine medical section information from a medical transcript is that most LLMs of necessity due to the way they operate with transformers and are trained will have a maximum input length of tokens (a way to measure how long the given transcript is). This input length is almost universally significantly shorter than the length of a standard medical interaction. To get around this the application uses three techniques:

All three of these methods first involve splitting the transcription into chunks that are shorter than the token limit.

In a first method, the method and system of the present invention will use the AI to write a note for each section as if the chunk were the entirety of the visit. Concatenate these notes into a single string and pass this string back into the LLM with the instruction to create a single note out of the many.

In a second method, the method and system of the present invention will use the AI to summarize the medically relevant portions of the chunks and then use the summarized medical portions as the input for the original AI to write the sections on the whole note.

In a third method, the method and system of the present invention will use the AI to extract the medically relevant information with regards to each section of the medical note i.e., “surgical history” from the encounter, and then use the original AI to write sections based on the chunked summaries.

In another alternative embodiment, a templating system can be incorporated into the present invention. The system of the present invention taught herein allows the user to select which sections of the note they would like for the AI (artificial intelligence) to write.

In still another alternative embodiment, the templating system and method consists of the user creating a new template with their own sections by going through a “template generator” wizard where they write which sections they would like for the note to contain, and give instructions to the AI for each section of the template. If the user prefers, this can also be accomplished by uploading a previously written note from another patient, and the AI used by the present invention will generate a template from this note in the same format as if the user had gone through the template generator wizard. This part is accomplished technically by giving an LLM instructions and/or training data around a specific template format, and then placing the desired template in the prompt along with the transcription and instructions to follow the template. This can be done with training or few-shot learning (known methods of getting LLMs to produce desired output systematically).

In yet another embodiment of the present invention, a template sharing system is incorporated into the present invention. In this embodiment, there is an online-hosted page in the website where users can try out other users' templates. A simple online library that organizes templates by the user's medical specialty and use-case is provided.

In still another embodiment, a lexicon system is incorporated into the present invention. This is a place where the user can input words and definitions that the AI tends to misinterpret or spell incorrectly. This is necessary for obscure drug names. The user inputs these items, and then the AI will add these into the LLM prompts to make sure that it is not mistaking any words that should be otherwise interpreted.

In another embodiment, a system to allow the user to speak to the AI and ask it to make edits to the note in its entirety is incorporated into the present invention. Instead of editing lines or portions of the note that the AI may have done incorrectly, the user is able to speak to the AI to have the AI make changes to the note.

Other technologies require installation of speakers while this application does not. Other systems do not allow the user to record on their phone and then view and change on their computer or vice versa.

This system and method taught by the present invention can be used for any profession that requires notes written about verbal encounters.

The software system is set to run on a computing device. A computing device on which the present invention can run would be comprised of a CPU, Hard Disk Drive, Keyboard, Monitor, CPU Main Memory and a portion of main memory where the system resides and executes. Any general-purpose computer with an appropriate amount of storage space is suitable for this purpose. Computer Devices like this are well known in the art and are not pertinent to the invention. The system can also be written in a number of different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Thus, it is appreciated that the optimum dimensional relationships for the parts of the invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A computer implemented method for using speech-to-text artificial intelligence to transcribe a doctor-patient interaction into a text format, the method comprising:
running and executing an application using speech-to-text artificial intelligence to record an audible doctor-patient interaction; and
running and executing an application using speech-to-text artificial intelligence to transcribe a doctor-patient interaction into a text format on an electronic device.

2. The method of claim 1, further comprising the recording steps of
providing a website or application on a computer, phone, or device for recording a doctor-patient interaction;
after logging in, the doctor will press “record” as he begins to interact with a patient; and
the doctor's device will record the interaction.

3. The method of claim 1, further comprising the speech-to-text steps of
using a speech-to-text artificial intelligence to transcribe the doctor-patient interaction into a text format;
switching on and off which speech-to-text AI that it uses; and
after the system of the present invention has received the transcription, it will ask the doctor what sections he would like in his medical note.

4. The method of claim 3, wherein the sections include:
Chief Complaint,
History of Present Illness,
Past Medical History,
Review of Systems,
Family History,
Social History,
Medication List,
Physical Examination/Objective section,
Assessment or Diagnosis,
Plan, and
Notes.

5. The method of claim 3, further comprising the speech-to-text steps of
after the doctor has made a selection of the pieces of the note he would like, the transcription of the recording between the doctor and patient is sent to the application server; and
the application server uses one or more a large-language model AI (LLM) to determine the content of any of the medical note sections.

6. The method of claim 5, further comprising the large language model (LLM) steps of
splitting the transcription into chunks that are shorter than the token limit of the large language model (LLM).

7. The method of claim 6, further comprising the large language model (LLM) steps of
using the AI to write a note for each section as if the chunk were the entirety of the visit;
concatenate these notes into a single string, and
pass this string back into the LLM with the instruction to create a single note out of the many.

8. The method of claim 6, further comprising the large language model (LLM) steps of
using the AI to summarize the medically relevant portions of the chunks, and
using the summarized medical portions as the input for the original AI to write the sections on the whole note.

9. The method of claim 6, further comprising the large language model (LLM) steps of
using the AI to extract the medically relevant information with regards to each section of the medical note from the encounter, and
using the original AI to write sections based on the chunked summaries.

10. The method of claim 1, further comprising a templating system enabling
selecting which sections of the note for the AI to write.

11. The method of claim 1, further comprising a templating system enabling
creating a new template from one or more existing user sections by going through a template generator wizard to
generate sections for the note to contain, and
give instructions to the AI for each section of the template.

12. The method of claim 11, wherein in the templating system
uploading a previously written note from another patient, the AI will generate a template from the previously written note in the same format as if a user had gone through the template generator wizard.

13. The method of claim 11, wherein in the templating system
accomplishes generating a template from the previously written note by
giving a large language model (LLM) instructions and/or training data around a specific template format, and
then placing the desired template in the prompt along with the transcription and instructions to follow the template.

14. The method of claim 10, further comprising a template sharing system wherein
there is an online-hosted page in the website where users can try out other users' templates; and
an online library that organizes templates by the user's medical specialty and use-case.

15. The method of claim 10, further comprising a lexicon system providing a place where the user can input words and definitions that the AI tends to misinterpret or spell incorrectly.

16. The method of claim 15, wherein
words and definitions are inputted using the lexicon system; and
the AI adds these into the LLM prompts to make sure that it is not mistaking any words that should be otherwise interpreted.

17. The method of claim 1, further comprising the speech-to-text steps of
speaking to the AI and asking it to make edits to the note in its entirety; and
speaking to the AI to have the AI make changes to the note.
